(19) United States
(12) Patent Application Publication
(10) Pub. No.: US 2007/0232909 A1
(43) Pub. Date: Oct. 4, 2007

(54) Ultrasonic Characterization of Internal Body Conditions Using Information Theoretic Signal Receivers

(75) Inventors: Michael S. Hughes, Wildwood, MO (US); Samuel A. Wickline, St. Louis, MO (US); Gregory M. Lanza, St. Louis, MO (US)

Correspondence Address: THOMPSON COBURN LLP;ATTN: RICHARD E. HAFERKAMP, ONE U.S. BANK PLAZA, SAINT LOUIS, MO, 63101, US

(73) Assignee: WASHINGTON UNIVERSITY, St. Louis, MO (US)

(21) Appl. No.: 11/692,706

(22) Filed: Mar. 28, 2007

Related U.S. Application Data
(60) Provisional application No. 60/786,750, filed on Mar. 28, 2006.

(51) Int. Cl. A61B 8/00

(52) U.S. Cl. 600/437

(57) ABSTRACT

Disclosed herein is a technique for performing medical imaging on a region of interest (ROI) wherein information theoretic signal receivers are used to enhance the detection and monitoring of pathologies such as angiogenesis and muscular dystrophy. Examples of information theoretic signal receivers that can be used in the practice of the invention include Shannon entropy signal receivers, continuous limit Shannon entropy signal receivers, Renyi entropy signal receivers, specific heat analog signal receivers, and thermodynamic energy analog signal receivers. The contrast enhancement provided by contrast agents, either targeted contrast agents or bubble-based contrast agents, is enhanced through the use of such information theoretic signal receivers. Further still, the contrast agents can be heated to further enhance visualization with information theoretic signal receivers.

### CROSS-REFERENCE AND PRIORITY CLAIM TO RELATED APPLICATION

This application claims priority to U.S. provisional patent application Ser. No. 60/786,750, entitled “System and Method for Ultrasonic Characterization of Internal Body Conditions Using Information Theoretic Signal Receivers”, filed Mar. 28, 2006, the entire disclosure of which is incorporated herein by reference.

### STATEMENT REGARDING FEDERALLY SPONSORED RESEARCH OR DEVELOPMENT

This invention was made with government support under NIH grants EB002168, RO1-HL042950, RO1-HL073646, U54-CA119342, and CO-27031 awarded by the National Institutes of Health (NIH). The government may have certain rights in the invention

### FIELD OF THE INVENTION

The field of this invention relates generally to the use of medical imaging to detect and diagnose internal body conditions. In a preferred embodiment, this technology relates to the use of ultrasound to achieve molecular imaging that allows noninvasive in vivo diagnosis of complex pathological processes such as angiogenesis.

### BACKGROUND AND SUMMARY OF THE INVENTION

Improvements in available medical imaging modalities coupled with advances in genomics and proteomics and the availability of microbubble-based, nanobubble-based and nanoparticle-sized nonbubble ultrasound contrast agents have allowed the vision of imaging and treating very small tumors to grow from a futuristic idea to a potential clinical tool. A primary goal of medical imaging is to enable the detection and tracking of a disease at the molecular level when gross morphological changes may not be present or apparent to conventional ultrasound methods. Contrast agents have been proposed to aid the diagnostic determination of disease with many imaging modalities, including positron emission tomography (PET), single-photon emission computed tomography (SPECT), optical imaging, magnetic resonance imaging (MRI), and ultrasound. With conventional clinical imaging systems known to the inventors herein, it is believed that the detection of cellular morphologic or functional abnormalities can take advantage of the presence of a contrast agent to amplify the pathological changes that are often below the resolution or detection threshold of the imaging modality.

For example, ultrasound has been used successfully as an imaging technology in the diagnosis of breast, liver, prostate, rectal and pancreatic cancers. Conventionally, diagnosis with ultrasonic imaging typically requires a change in gross morphology of the underlying organ or a change in function such as perfusion or blood velocity. In the case of breast cancer, the size, shape, shadowing, internal echogenicity, lobulations, and other factors indicate the presence of a suspicious lesion. Commonly, the lesions are then biopsied for ultimate confirmation of whether they are malignant or benign. Because many cancers can only be diagnosed by ultrasonic interrogation if those cancers are sufficiently extensive to alter the surrounding architecture, it is believed by the inventors herein that conventional ultrasonic imaging may overlook earlier stage cancers.

As noted above, the use of contrast agents is a known technique to enhance the detection of cancers in conjunction with ultrasonic imaging. For example, non-targeted microbubble-based ultrasonic contrast agents have been used to assist the detection of cancer in the liver, prostate, and breast. These contrast agents permit determination of the perfusion properties of normal and cancerous tissue through the observation of wash-in and wash-out curves, and through identification of telltale vasculature in unusual anatomic locations that are highlighted by the perfusing bubbles.

One molecular strategy for identifying solid cancers involves specific detection of molecular markers of tumor angiogenesis. In 1971, Judah Folkman proposed that the energetics of new cell growth associated with cancer demanded an energy source. Since means of delivering energy to an removing waste products from cancer cells are compromised near the pathology, especially when the tumor cells reach sizes greater than 1-2 mm³, it is believed that there must be some mechanism for cancer cells to alter their surrounding architecture to encourage new vessel growth. Previous studies have demonstrated an increase in new vessel growth ostensibly simulated by hypoxia in rapidly multiplying cancer cells that accompanies an “angiogenic switch” to a more aggressive phenotype capable of metastasis. The recruitment and formation of new vessels from surrounding vessels, otherwise known as angiogenesis, can provide a means of detecting early stage cancer by targeting angiogenic vessels or expressions of signaling and structural proteins associated with new vessel growth. A variety of proangiogenic signaling proteins (bFGF, VEGF, tissue factor, hypoxia inducing factor, αᵥβ₃ integrins, etc.) have been examined as part of the cascade of reactions occurring to encourage new vessel growth. The integrin αᵥβ₃ has been implicated in the migration and proliferation of endothelial cells associated with angiogenesis. The inventors herein have previously shown that the expression of αᵥβ₃ appears especially prominent in areas of neovascularization and that other pathological situations can result in upregulation through local inflammatory responses such as balloon angioplasty. Although αᵥβ₃ integrins may not be mandatory for angiogenesis, it is believed that they are excellent biomarkers that appear in the neighborhood of early vessel formation and have been associated with a propensity to metastasis in breast and other tumors as a correlated function of increased microvascular

For site-targeted nanoparticles applied at frequencies above 25 MHz, conventional ultrasonic backscatter as processed using known proprietary methods by ultrasound imaging devices has been shown to be sensitive to the presence of targeted nanoparticles, both in vitro and in vivo, especially when the targets of the nanoparticles are abundant. Theoretical modeling indicates that these results may be understood to a rough first order approximation in terms of a simple transmission line model.

Effective molecular imaging with ultrasound is highly desirable because ultrasound technology is clinically ubiquitous throughout the world, and because ultrasound technology is a comparatively cheap, portable, and straightforward imaging modality that is available at most medical centers. Accordingly, ultrasound technology offers an opportunity to implement clinical molecular imaging with global penetration as well as relevance to broad-based disease segments.

However, at lower ultrasound frequencies such as those available with most clinical ultrasound imaging equipment (e.g., frequencies in the range of approximately 2 MHz to approximately 15 MHz), it is generally believed that nanoparticle detection is less sensitive than clinical frequency bubble detection, especially for sparse concentrations of targets found in early cancers. This is cause for concern as effective detection techniques at clinical ultrasound frequencies (relative to higher frequency research models) are desirable to more fully take advantage of ultrasound's global reach. Accordingly, the inventors herein believe that a need exists to look beyond the use of conventional ultrasound imaging techniques as a way to enhance the detection of contrast agent-enhanced internal body conditions (such as pathologies) with a wider array of ultrasound imaging systems.

In an effort to fill this need in the art, the inventors herein disclose the use of information theoretic signal receivers as a post-processing adjunct to ultrasound imaging in not only high frequency ranges, but also lower frequency ranges such as those often employed in most clinical settings. Preliminary testing by the inventors herein have shown unexpected results in that detection of contrast-agent enhanced internal body conditions such as angiogenesis can be achieved at clinical ultrasound frequencies when information theoretic signal receivers are applied to the RF waveform produced by the ultrasound transducer. Furthermore, the inventors herein believe that even at higher frequencies, information theoretic signal receivers exhibit sensitivity that is superior to conventional signal processing techniques. Investigation by the inventors has also indicated that the detection and monitoring of tissue affected by muscular dystrophy can be enhanced when information theoretic signal receivers are applied to the RF waveform produced by the ultrasound transducer.

A “signal receiver”, as used herein, refers to an algorithm that is configured to transform an RF waveform or a portion thereof into a single datum (e.g., value or number). Such an algorithm can be implemented on a variety of devices (e.g., using software to be executed on a processor, using dedicated or reprogrammable hardware, or some combination thereof). The device that carries out this algorithm may be internal to or external to the ultrasound imaging system that is used to obtain the raw ultrasound RF waveform data. Examples of signal receivers that are used in conventional ultrasound applications include total energy signal receivers, log of total energy signal receivers, and log of total energy signal receivers.

Previous work by one of the inventors herein led to the development of “information theoretic” signal receivers. See U.S. Pat. Nos. 5,247,302, 5,280,291, and 5,392,046, the entire disclosures of each of which are incorporated herein by reference; see also Hughes, Michael S., A Comparison of Shannon entropy versus signal energy for acoustic detection of artificially induced defects in Plexiglas, J. Acoust. Soc. Am. 91(4), Pt. 1, pp. 2272-75, April 1992; Hughes, Michael S., Analysis of Ultrasonic Waveforms Using Shannon Entropy, 1992 Ultrasonics Symposium, pp. 1205-09, 1992; Hughes, Michael S., Analysis of digitized waveforms using Shannon entropy, J. Acoust. Soc. Am., 93(2), pp. 892-906, February 1993; Hughes, Michael S., Analysis of digitized waveforms using Shannon entropy. II. High-speed algorithms based on Green's functions, J. Acoust. Soc. Am., 95(5), Pt. 1, pp. 2582-88, May 1994, the entire disclosures of each of which are disclosed herein by reference. As used herein, an “information theoretic signal receiver” refers to a signal receiver that computes a statistical distribution of digitized voltage values from at least a portion of a waveform signal and then computes the single datum for the values of the computed distribution using a thermodynamics-based formula.

Experimentation by the inventors herein has lead the inventors to conclude that the combination of information theoretic signal receivers with the administration of contrast agents provides marked enhancements to the detectability of internal pathologies such as angiogenesis. The inventors herein believe that these improvements arise for a number of reasons. For example, unlike conventional ultrasound signal receivers (which are highly dependent upon the magnitude of the RF signal), information theoretic signal receivers are sensitive to the shape, i.e., the undulations, of the RF waveform and are less signal-to-noise dependent. Moreover, information theoretic signal receivers are capable of effectively operating without the gating of input RF data, thereby providing an opportunity to eliminate operator dependence on results. Further still, because information theoretic signal receivers represent a post-processing step, the present invention need not require the design and construction of new image acquisition systems. Moreover, the inventors herein believe that information theoretic signal receivers function well when the scattering object is near an interfacial boundary, which can be a significant issue in connection with the detection of angiogenesis, where tumor neovasculature vessels often bridge from an adjacent tissue border into the developing tumor capsule.

The inventors herein also disclose that the application of heat to the contrast agent in conjunction with the use of information theoretic signal receivers can improve contrast enhancement for ultrasound imaging. This heat is preferably a transient heat exhibiting a temperature that is nonlethal for the cells subjected to the imaging. Further still, this transient heating preferably produces no cavitation (or negligible amounts of cavitation) in the region subject to the imaging.

Further still, the inventors disclose herein numerous potential applications for the inventive methodology, including but not limited to detecting pathologies, monitoring tissue change over time, enhancing contact-facilitated drug delivery, and monitoring the effectiveness of contact-facilitated drug delivery.

As an exemplary embodiment of the invention, the inventors herein disclose a method for imaging a region of interest (ROI) within a body to obtain an image indicative of a condition of muscular dystrophy affecting the ROI, the method comprising: (1) applying ultrasound energy to the ROI; (2) acquiring raw ultrasound data for the ROI in response to the applied energy; and (3) applying an information theoretic signal receiver to the acquired raw ultrasound data to thereby generate information theoretic data from which an ultrasound image of the ROI can be created, wherein the ultrasound image is indicative of a condition of muscular dystrophy affecting the ROI. The ROI may comprise tissue that has been treated with a steroid. Also, the method may further comprise repeating the method steps over time to determine a progressive condition of the muscular dystrophy affecting the steroid-treated tissue over time. The repeating step may be performed without requiring exact matching of previous settings for an ultrasound imager when it performs the applying and raw ultrasound data acquiring steps. The method may also further comprise: (1) applying an energy-based signal receiver to the acquired raw ultrasound data to thereby generate data from which another ultrasound image of the ROI can be created; and (2) determining the progressive condition of the muscular dystrophy affecting the steroid-treated tissue based on a combination of the information theoretic-based ultrasound image and the energy-based ultrasound image. Moreover, the applying step may comprise applying ultrasound energy to the ROI wherein the applied ultrasound energy has a frequency in a range of approximately 2 MHz to approximately 15 MHz.

As another exemplary embodiment of the invention, the inventors disclose a method for imaging a region of interest (ROI) within a volume to obtain an image indicative of whether a condition of interest exists within the volume, the method comprising: (1) injecting the region of interest with a contrast agent that is targeted to the condition of interest; (2) applying ultrasound energy to the ROI; (3) acquiring ultrasound data for the ROI in response to the applied energy; and (4) applying an information theoretic signal receiver to the acquired ultrasound data to thereby generate information theoretic data from which an ultrasound image of the ROI can be created, wherein the ultrasound image is indicative of whether the condition of interest exists within the volume. The step of applying ultrasound energy may comprise applying ultrasound energy to the ROI wherein the applied ultrasound energy has a frequency in a range of approximately 2 MHz to approximately 15 MHz. The step of applying ultrasound energy may also comprise applying ultrasound energy to the ROI wherein the applied ultrasound energy has a frequency in a range of approximately 7 MHz to approximately 15 MHz. Further, the step of applying ultrasound energy may also comprise applying ultrasound energy to the ROI wherein the applied ultrasound energy has a frequency in a range of approximately 2 MHz to approximately 4 MHz. As an example, the condition of interest may comprise angiogenesis (including angiogenesis corresponding to a tumor). As another example, the condition of interest may comprise tissue affected by muscular dystrophy. The method may further comprise repeating the ultrasound energy applying step, the acquiring step and the information theoretic signal receiver applying step over time to monitor how the condition of interest progresses over time. As an example, the contrast agent may comprise an αᵥβ₃-targeted contrast agent. Optionally, this αᵥβ₃-targeted contrast agent can be loaded with a therapeutic drug for contact-facilitated drug delivery, and wherein the repeating step thereby allows the drug delivery to be monitored over time. Further still, the αᵥβ₃-targeted contrast agent may comprise a plurality of αᵥβ₃-targeted lipid-encapsulated, perfluorocarbon nanoparticles. As another example, the contrast agent may comprise a bubble-based contrast agent. The acquiring step may comprise receiving raw ultrasound data in response to the applied ultrasound energy and upsampling the raw ultrasound data to stabilize the information theoretic signal receiver when it is applied to the acquired ultrasound data. Further still, the acquiring step may comprises performing a moving window analysis on the upsampled data to generate a plurality of windows of upsampled data. Furthermore, the information theoretic signal receiver applying step may comprise applying an information theoretic signal receiver to each generated window of upsampled data. It should also be noted that the step of applying an information theoretic signal receiver may comprise at least one member selected from the group consisting of (1) applying a Shannon entropy (Hₛ) signal receiver to the acquired ultrasound data to thereby generate an ultrasound image that is indicative of whether the condition of interest exists within the volume, (2) applying a continuous limit Shannon entropy (H_{c}) signal receiver to the acquired ultrasound data to thereby generate an ultrasound image that is indicative of whether the condition of interest exists within the volume, (3) applying a Renyi entropy (Z) signal receiver to the acquired ultrasound data to thereby generate an ultrasound image that is indicative of whether the condition of interest exists within the volume, (4) applying a specific heat analog (Cᵥ) signal receiver to the acquired ultrasound data to thereby generate an ultrasound image that is indicative of whether the condition of interest exists within the volume, and (5) applying a thermodynamic energy analog (Eₜₕ) signal receiver to the acquired ultrasound data to thereby generate an ultrasound image that is indicative of whether the condition of interest exists within the volume. It should also be noted that the method may further comprise heating at least a portion of the ROI after the contrast agent has been injected and prior to the ultrasound data acquiring step. Further, when the ROI comprises a region within a patient's body having a plurality of cells, and the heating step may comprise heating the ROI to a temperature that is nonlethal to the cells. Moreover, the heating step may comprise applying the heat to the ROI with a heating pulse of ultrasound energy or microwave energy. Also, the information theoretic data preferably comprises a plurality of pixels having data values, and the method may further comprise: (1) defining a threshold for the information-theoretic data; (2) selecting pixels of the information theoretic data whose data values are equal to or greater than the threshold; and (3) generating the ultrasound image by combining the acquired ultrasound data with the selected pixels to create a hybrid ultrasound image.

As another exemplary embodiment of the invention, the inventors disclose a method for imaging a region of interest (ROI) within a volume to obtain an image indicative of whether a condition of interest exists within the volume, the method comprising: (1) injecting the ROI with a contrast agent, the contrast agent comprising a plurality of bubbles; (2) applying a first ultrasound energy to an area within the ROI, wherein the ultrasound energy has sufficient power to eliminate substantially all of the bubbles from the area; (3) after the area has refilled with a plurality of wash-in bubbles, applying a second ultrasound energy to the area within the ROI, wherein the ultrasound energy has a sufficiently low power to not eliminate the wash-in bubbles; (4) acquiring raw ultrasound data for the ROI in response to the applied second ultrasound energy; and (5) applying an information theoretic signal receiver to the acquired raw ultrasound data to thereby generate an ultrasound image that is indicative of whether the condition of interest exists within the volume. Preferably, for this embodiment, the contrast agent comprises a microbubble-based contrast agent or a nanobubble-based contrast agent. The step of applying the second ultrasound energy may comprise applying second ultrasound energy to the ROI wherein the applied second ultrasound energy has a frequency in a range of approximately 7 MHz to approximately 15 MHz. The step of applying the second ultrasound energy may also comprise applying second ultrasound energy to the ROI wherein the applied second ultrasound energy has a frequency in a range of approximately 2 MHz to approximately 4 MHz. Furthermore, the acquiring step may comprise: (1) receiving raw ultrasound data in response to the applied second ultrasound energy, (2) upsampling the raw ultrasound data to stabilize the information theoretic signal receiver when it is applied to the acquired ultrasound data, and (3) performing a moving window analysis on the upsampled data to generate a plurality of windows of upsampled data, and wherein the information theoretic signal receiver applying step comprises applying an information theoretic signal receiver to each generated window of upsampled data.

As another exemplary embodiment of the invention, the inventors disclose a system for detecting whether a condition exists within a body, the system comprising: (1) a delivery device for delivering a contrast agent to a region of interest (ROI) within the body; (2) a medical imaging device for producing, after the contrast agent has been delivered to the ROI via the delivery device, raw data from which an image of the ROI can be generated; and (3) a processor in communication with the medical imaging device, the processor being configured to (a) receive the raw data from the medical imaging device, and (b) process the raw data through an information theoretic signal receiver to thereby generate processed data indicative of whether the condition exists. Preferably, the medical imaging device comprises an ultrasound imaging device. Also, the contrast agent may comprise a targeted contrast agent, which optionally may be loaded with a therapeutic drug for contact-facilitated drug delivery. The targeted contrast agent may comprise an αᵥβ₃-targeted contrast agent, such as a plurality of αᵥβ₃-targeted lipid-encapsulated, perfluorocarbon nanoparticles. Also, the condition may comprise a condition such as angiogenesis. Moreover, the ultrasound imaging device can be configured to deliver an acoustic wave to the ROI having a frequency in a range of approximately 2 MHz to approximately 15 MHz. The ultrasound imaging device can also be configured to deliver an acoustic wave to the ROI having a frequency in a range of approximately 2 MHz to approximately 7 MHz. Moreover, the ultrasound imaging device can be further configured to heat at least a portion of the ROI such that the ROI portion is heated to a temperature that is nonlethal for a majority of cells therein when the raw data is produced, thereby enhancing the detectability of the condition. The information theoretic signal receiver may comprise at least one selected from the group consisting of (1) a Shannon entropy signal receiver, (2) a continuous limit Shannon entropy signal receiver, (3) a Renyi entropy signal receiver, (4) a specific heat analog signal receiver, and (5) a thermodynamic energy analog signal receiver. Moreover, the processor can be further configured to generate a displayable image at least partially from the processed data, the image being indicative of whether the condition exists.

As yet another exemplary embodiment of the invention, the inventors disclose, a method for visualizing an internal condition within a body, the method comprising: (1) intravascularly administering a nanoparticle contrast agent into a region of interest (ROI) within the body, wherein the nanoparticle contrast agent is targeted to a condition of interest; (2) heating the ROI; (3) applying ultrasound energy to the heated ROI to generate ultrasound data; and (4) applying the ultrasound data to an information theoretic signal receiver to thereby generate image data indicative of whether the condition of interest exists within the ROI. The heating step may comprise heating the ROI to a temperature that is nonlethal for a majority of cells within the ROI. The heating step may further comprise heating the ROI to induce a temperature change in the ROI that falls within a range of about 1 to 10 degrees Centigrade. The heating step may also comprise heating the ROI such that cavitation in the ROI is avoided. Further still, the heating step may comprise delivering a heating pulse to at least a portion of the ROI via ultrasound or via microwave. The targeted nanoparticle contrast agent may comprise an αᵥβ₃-targeted nanoparticle contrast agent, which may comprise a plurality of αᵥβ₃-targeted lipid-encapsulated, perfluorocarbon nanoparticles. The condition of interest may comprise a condition such as angiogenesis. The step of applying ultrasound energy to the heated ROI may comprise applying an acoustic wave to the ROI having a frequency in a range of approximately 2 MHz to approximately 15 MHz. The step of applying ultrasound energy to the heated ROI may also comprise applying an acoustic wave to the ROI having a frequency in a range of approximately 2 MHz to approximately 5 MHz. It should also be noted that method can be performed in vivo. Further still, the generated image data may comprise a plurality of pixels having pixel values, and the method may further comprise: (1) thresholding the generated image data to thereby select a subset of the generated image data's pixels; (2) generating a plurality of pixels for an ultrasound image from the ultrasound data; (3) combining the selected subset of pixels with the pixels generated from the ultrasound data to create an ultrasound image that is indicative of whether the condition of interest exists within the ROI. The method may also further comprise color coding the selected subset of pixels.

These and other features and advantages of the present invention will be in part apparent and in part pointed out hereinafter to those having ordinary skill in the art with reference to the following description, claims and figures.

### BRIEF DESCRIPTION OF THE DRAWINGS

FIG. 1 depicts an exemplary overview of an imaging system capable of implementing the present invention;

FIG. 2 depicts an exemplary flowchart that describes a methodology of a preferred embodiment of the present invention;

FIG. 3 depicts an exemplary perfluorocarbon nanoparticle emulsion;

FIG. 4 depicts an exemplary cumulative distribution function histogram with upper 5% thresholding;

FIG. 5 depicts an exemplary flowchart that describes a methodology for a microbubble-based contrast agent imaging embodiment;

FIG. 6 depicts an exemplary flowchart that describes a methodology for an embodiment wherein heating is used to enhance performance;

FIG. 7 depicts a plot of contrast enhancement as a function of temperature for an information theoretic signal receiver and conventional signal receivers;

FIG. 8 illustrates “contact-facilitated” drug delivery that can be monitored through the use of the present invention; and

FIG. 9 illustrates images produced in accordance with the present invention showing contrast enhancement of targeted areas corresponding to a VX-2 tumor in a New Zealand white rabbit.

### DETAILED DESCRIPTION OF THE PREFERRED EMBODIMENT

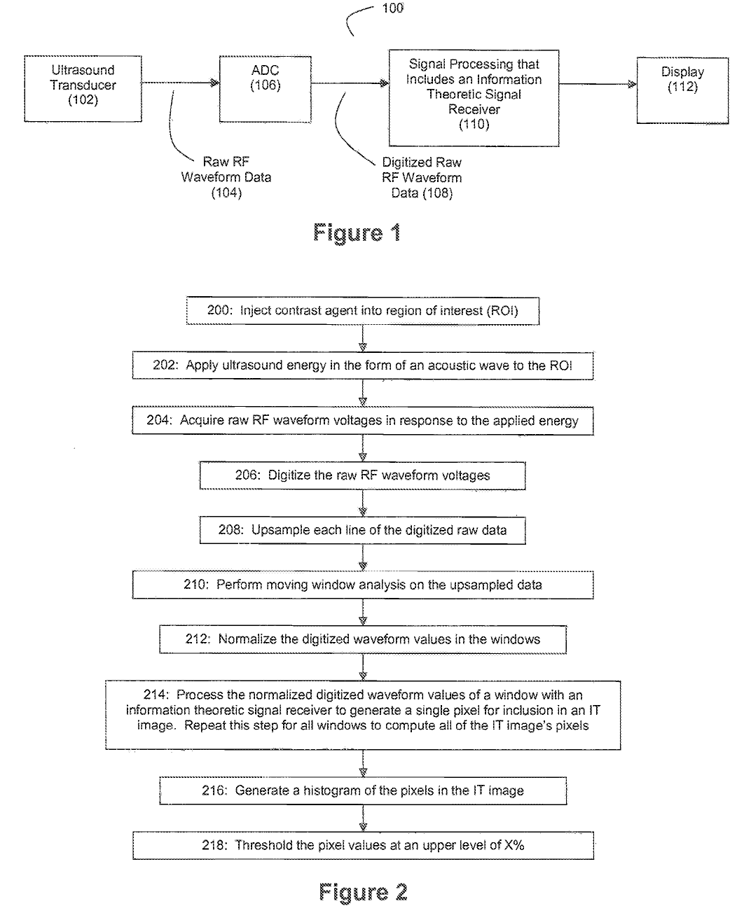

FIG. 1 depicts an exemplary overview of an imaging system 100 capable of implementing the present invention. Preferably, the imaging system 100 is an ultrasound imaging system. However, other imaging modalities may be used in the practice of the present invention, including but not limited to magnetic resonance imaging and nuclear imaging. However, as set forth herein, the inventors believe that the present invention is particularly amenable to implementation with ultrasound imaging, both in vivo and in vitro. Additionally, the techniques of the present invention can be applied to areas such as intravascular imaging, transesophageal imaging, and acousto-optic imaging.

An ultrasound transducer 102 operates to apply an acoustic signal to a region of interest (ROI) and receive acoustic reflections that are transformed into raw RF waveform data 104 that is representative of the interaction of the acoustic signal with the ROI. This raw RF waveform data 104 is then processed through an analog-to-digital converter (ADC) 106 that digitizes the raw RF waveform produced by the transducer 102.

In one exemplary embodiment, the ultrasound system can be a research imager such as a Vevo 660 ultrasound system available from Visualsonics of Toronto, Canada with a 30 MHz single element “wobbler” probe/transducer configured to acquire the raw waveform data in a B-scan format, as is known in the art. However, any of a number of commercially-available or custom-designed ultrasound acquisition systems can be used in the practice of the present invention, including clinical ultrasound systems that operate at lower frequencies than do higher end research models. The ultrasound transducer 102 preferably acquires beam-formed RF data in a B-scan format at select times (e.g., pre-injection and at 0, 15, 30, 60, and 120 minutes after injection of a contrast agent into the area of interest).

The ADC 106 operates to sample the raw waveform 104 at a predetermined sampling rate (such as 500 MHz) to generate a digitized waveform 108 that comprises multiple frames, each of which comprising a plurality of lines of numerous multi-bit words. Each frame corresponds spatially to an area within the ROI.

A processor 110 that includes an information theoretic signal receiver then operates to receive and process the digital data 108 to compute pixel values. Thereafter, images corresponding to the computed pixel values can be shown on a display 112 such as the screen of a computer monitor or the like. The inventors believe that such an image will be capable of indicating whether a condition of interest exists within the ROI with greater precision than conventional ultrasound images that are processed using the conventional total energy or log(total energy) signal receivers that are known in the art. Examples of conditions of interest that can be gauged by the practice of the present invention include angiogenesis (in various conditions such as solid tumor growth (VX2 tumors, MDA435 tumors, etc.) and metastasis, diabetic retinopathy, macular degeneration, muscular dystrophy or atherosclerosis due to expansion of the vasa vasorum under high cholesterol drive), blood clots (in arteries, veins, heart chambers, aorta or on valves), small deposits of fibrin or platelets on unstable atherosclerotic plaque, and small collections of tumor cells in accessible parts of the body to name just a few. The processor 110 can be any commercially-available processor having sufficient computing power to carry out the operations described herein.

Examples of information theoretic signal receivers that can be used in the practice of the present invention are those described U.S. Pat. Nos. 5,247,302, 5,280,291, 5,392,046 and the publications Hughes, Michael S., A Comparison of Shannon entropy versus signal energy for acoustic detection of artificially induced defects in Plexiglas, J. Acoust. Soc. Am. 91(4), Pt. 1, pp. 2272-75, April 1992; Hughes, Michael S., Analysis of Ultrasonic Waveforms Using Shannon Entropy, 1992 Ultrasonics Symposium, pp. 1205-09, 1992; Hughes, Michael S., Analysis of digitized waveforms using Shannon entropy, J. Acoust. Soc. Am., 93(2), pp. 892-906, February 1993; Hughes, Michael S., Analysis of digitized waveforms using Shannon entropy. II. High-speed algorithms based on Green's functions, J. Acoust. Soc. Am., 95(5), Pt. 1, pp. 2582-88, May 1994, particular examples of which include entropy signal receivers (e.g., Shannon entropy (Hₛ), continuous limit Shannon entropy (H_{c}), and Renyi entropy (Z) signal receivers), specific heat analog (Cᵥ) signal receivers, and thermodynamic energy analog (Eₜₕ) signal receivers.

FIG. 2 is a flowchart that illustrates a preferred methodology for the present invention. At step 200, a contrast agent is injected into the ROI. Preferably, the contrast agent is injected intravascularly via a delivery device such as a catheter or needle. The contrast agent can be a nongaseous contrast agent that is targeted to a condition of interest or it can be a microbubble-based or nanobubble-based contrast agent.

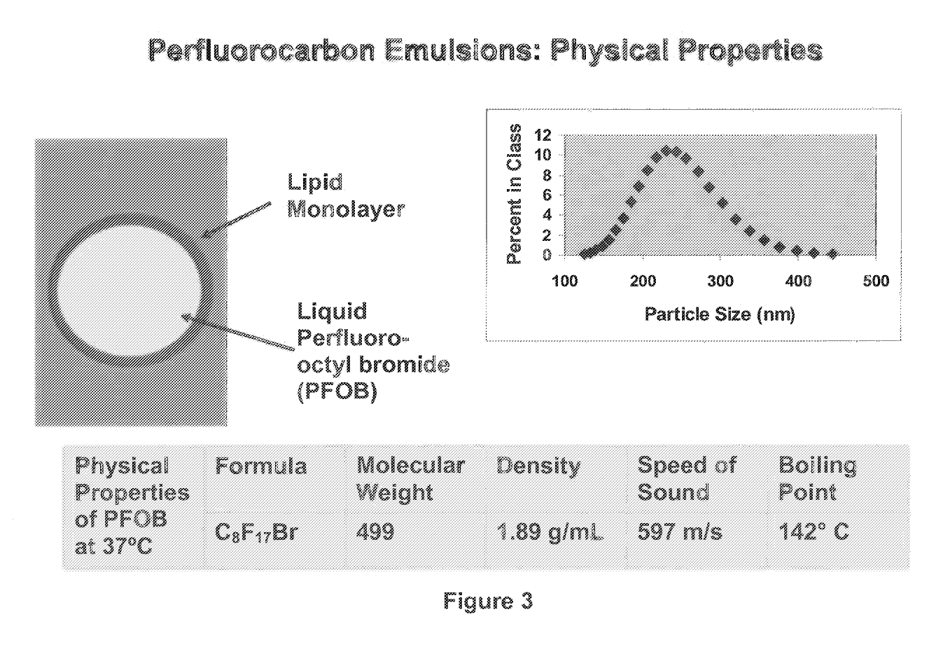

With respect to targeted contrast agents, examples include contrast agents that are targeted to αᵥβ₃ in angiogenic vessels, contrast agents that are targeted to fibrin in atherosclerotic placques, contrast agents that are targeted to fibrin in thrombi, and contrast agents that are targeted to tissue factor in blood vessel walls or in tumors. A preferred targeted contrast agent for use in the present invention comprises lipid-encapsulated, liquid perfluorocarbon nanoparticles that are approximately 200-250 nm in diameter, as shown in FIG. 3 and as described in previous works by the inventors such as Lanza, G. and Wickline, S., Targeted Ultrasonic Contrast Agents for Molecular Imaging and Therapy, Process in Cardiovascular Diseases, 44(1), pp. 13-31, 2001, Lanza et al., In situ localization of tissue-factor following carotid angioplasty using a ligand-targeted ultrasonic contrast agent and intravascular ultrasound, Journal of American College of Cardiology, 31(1), p. 19, 1998; Lanza et al., In vivo molecular imaging of stretch-induced tissue factor in carotid arteries with ligand targeted nanoparticles, Journal of the American Society of Echocardiography, 44, pp. 433-39, 2000; Hall et al., Time evolution of enhanced ultrasonic reflection using a fibrin-targeted nanoparticle contrast agent, Journal of the Acoustical Society of America, 108(6), pp. 3049-57, 2000; Anderson et al., Magnetic resonance contrast enhancement of neovasculature with alpha/sub v/beta/sub 3/-targeted nanoparticles, Magnetic-Resonance-in-Medicine, 44(3), pp. 433-39, 2000, U.S. Pat. Nos. 5,690,907, 5,780,010, 5,958,371, 6,548,046, and 6,676,963, and published U.S. patent applications 2003/0086867A1, 2004/0058951A1, and 2004/0248856A1, the entire disclosures of each of which are incorporated herein by reference. The nanoparticles can be loaded with gadolinium to achieve MRI contrast, while the perfluorocarbon liquid interior of the agent simultaneously provides acoustic contrast. These particles have been successfully targeted to a variety of molecular markers for imaging with either or both ultrasound and MRI as explained in the above-cited articles and patents.

With respect to microbubble-based contrast agents, the inventors herein believe that ultrasound imaging in accordance with the present invention can be performed with fewer constraints than with conventional signal receivers. With conventional signal receivers, ultrasound imaging in conjunction with microbubble-based contrast agents typically requires that the ultrasound energy be tuned to a particular frequency based on microbubble size. However, when information theoretic signal receives are used on the ultrasound data, the inventors herein believe that reliable and accurate results can be obtained with ultrasound imaging frequencies that are independent of the microbubble size, which will make microbubble-based ultrasound imagery easier to perform and may reduce potential frequency-related side effects.

At step 202, the ultrasound transducer 102 is preferably used to apply ultrasound energy in the form of an acoustic wave to the ROI. Because of the sensitivity provided by the information theoretic signal receivers, this energy can be applied at frequencies much lower than the high “research” ultrasound frequencies(e.g., 25 MHz or above). When information theoretic signal receivers are used in conjunction with targeted contrast agents and ultrasound imaging to detect conditions such as angiogenesis, the inventors herein believe that effective imaging results can be obtained at intermediate frequencies (e.g. 7-15 MHz) with clinical imaging equipment, and even at low frequencies (e.g., 2-4 MHz) with clinical imaging equipment. The inventors posit that the ability to effectively image conditions such as angiogenesis at lower frequencies with the techniques of the present invention arises from information theoretic signal receivers' sensitivity to diffuse, low amplitude features of the RF signal that are often otherwise obscured by noise or hidden in large specular echoes.

At step 204, the ultrasound transducer 102 preferably senses the reflected acoustic wave and produces as an output the raw RF waveform voltages 104 from which the images will be produced. At steps 202 and 204, it is preferred that the ultrasound transducer operate in B-scan mode. However, it should be noted that A mode and M mode data can be used. Furthermore, the ultrasound transducer 102 can be any of a variety of known ultrasound transducer configurations, including a single transducer, an array of transducers , and a wobbler medical imaging probe.

At step 206, as noted above, the ADC 106 preferably samples the raw waveform at a predetermined sampling rate (such as 500 MHz) to generate digitized raw RF waveform data 108 that comprises a plurality of frames, each frame comprising a plurality of lines, each line comprising a plurality of multi-bit words whose values correspond to pixel values. For example, a frame may comprise 384 lines of 4096 8-bit words/pixels. However, it should be readily understood that other frame sizes could be used in the practice of the invention.

At step 208, the digitized waveform data 108 is upsampled in order to stabilize the information theoretic signal receiver algorithms, which benefit from increased waveform length. With the 384 line/4096 word example above, each of the 384 lines of data can be upsampled from 4096 words to 8192 words. Preferably, this upsampling uses a cubic spline that is fit to the original data set 108. However, it should be readily understood that other upsampling techniques could be used in the practice of the invention.

At step 210, a moving window analysis is performed on the upsampled data. With a moving window analysis, (1) a plurality W of consecutive points (which can also be referred to as words or pixels) in the waveform are selected starting from the first point to generate the first window position,(2) another W consecutive points in the waveform are selected starting from point S in the waveform to generate the second window position, (3) another W consecutive points in the waveform are selected starting from point 2S in the waveform to generate the third window position, and (4) this process is repeated until it is no longer possible to select W consecutive points in the waveform data without going past the last point in the waveform data. The W consecutive points can be referred to as the windowed points (or words/pixels) since they may be thought of as having been obtained by applying a square wave window to the entire digital waveform and taking the nonzero points of the resulting windowed waveform. As this selection of windowed points is made at successively greater delays into the entire digital waveform, this process may be thought of as having been performed by sliding a square gating function over the entire digital waveform, hence the term sliding window analysis. Exemplary values for W and S are 64 and 32 respectively, but it should be readily understood that other positive integer values for W and S could be used in the practice of the present invention (e.g., particularly other powers of 2). For example with the 384 line/8192 word example used herein, this moving window analysis can use a window having a word length W of 64 words that is moved in 0.064 μs steps (or a stepsize S of 64 words), thereby resulting in 128 window positions within the original data set.

Next at step 212, the pixel values in the windows are normalized or scaled to a unitless quantity. To normalize the pixel values to a unitless quantity, the following formula can be used:

Normalized Pixel Value=(Original Pixel Value−m)/σ

wherein m is the mean of the original pixel values and wherein σ is the standard deviation of the original pixel values. Preferably, the values of the words in each window are scaled or normalized using the same scaling or normalization technique prior to the application of the information theoretic signal receiver to each window position. It should be noted that this step can also be performed prior to step 210.

Next, at step 214, the normalized values of the windowed words within each window position are applied to an information theoretic signal receiver to compute the single datum for each window position. Each datum then serves as a pixel in a resulting information theoretic (IT) image. Thus the IT image for the 384 line/8192 word example would have 128 pixels whose values are the single values computed by the information theoretic signal receiver for each of the window positions.

As mentioned above, preferred information theoretic signal receivers include Hₛ signal receivers, H_{c} signal receivers, Z signal receivers, Cᵥ signal receivers, and Eₜₕ signal receivers. While the implementation details of such information theoretic signal receivers are known in the art from the patents and publications cited above and incorporated by reference herein, a brief summary of this technology will be reiterated.

Information theoretic signal receivers analyze the statistical distribution of digitized voltage levels from an acoustic signal and are highly sensitive to diffuse, low amplitude features of the signal that are often obscured by noise or lost in large specular echoes.

A starting point for the information theoretic signal receiver is an acoustic waveform that has been digitized into discrete voltage levels on a discrete time lattice. From this, a normalized histogram of the voltage levels of the digitized waveform is used to compute the Shannon entropy:

$${H}_{S} = {\sum }_{i = 1}^{{N}_{α}} {p}_{i} ln ( {p}_{i} )$$

The limiting form of Shannon entropy, the continuous limit Shannon entropy (H_{c}), is also useful as an information theoretic signal receiver. Further still, significant sensitivity may be gained by exploiting analogies to thermodynamics to derive a partition function:

$$Z ( β ) = {\sum }_{i} {e}^{- {βɛ}_{i}}$$

where the {εᵢ}_{i=0, . . . , N}_{_{α}}₋₁ are related to the original probabilities by

$${p}_{i} = \frac{{e}^{- {β}_{0} {ɛ}_{i}}}{{\sum }_{i} {e}^{- {β}_{0} {ɛ}_{i}}}$$

From the partition function, one can readily compute other thermodynamic quantities such as the partition function itself (Z(β)), the entropy (Hₛ(β)), the specific heat (Cᵥ(β)) and the thermodynamic energy (Eₜₕ(β)), the details of which are described in the above-cited and incorporated references.

Optionally, further processing can be performed on the IT image. For example, next, this process can include the step 216 of creating a histogram of the pixels in the IT image to obtain the probability density function (PDF).

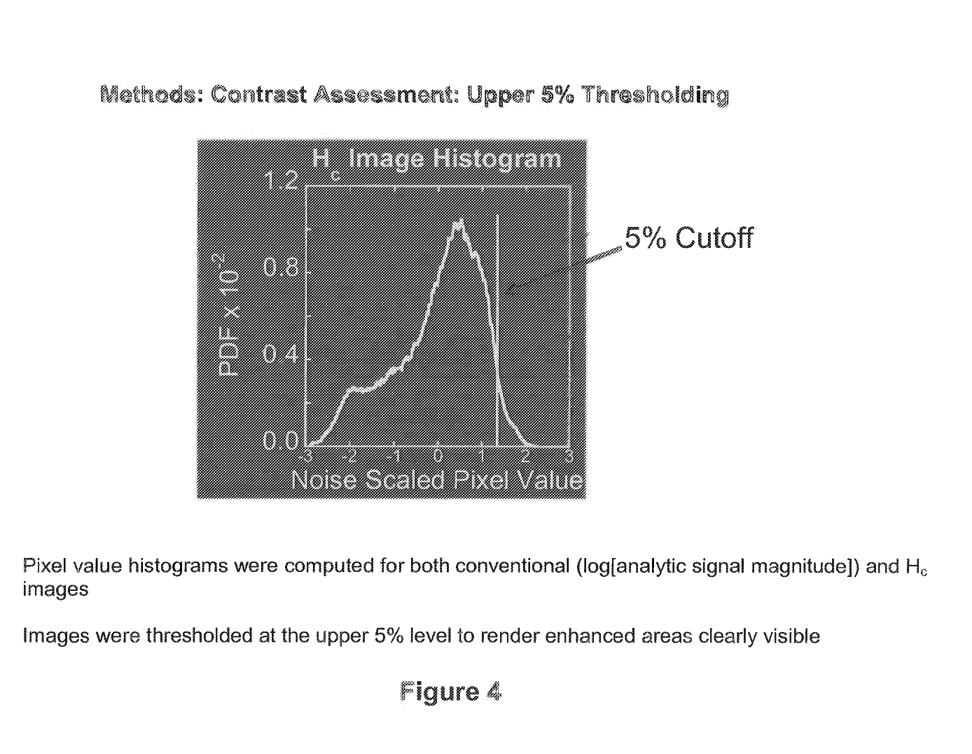

Next, at step 218, the PDF data is integrated to obtain the cumulative distribution function (CDF), as shown in FIG. 4. The pixels in CDF can then be thresholded at some percentage (such as the upper 5% of normalized pixel values) to segment the image into regions corresponding to targeted areas and non-targeted areas. The choice of a specific threshold value is a design parameter that can be varied by practitioners of the invention to suit a given application. Moreover, pixels falling above or below the threshold can be color-coded in the image to render targeted areas more clearly. Further still, the use of such a CDF analysis of normalized pixel values enables a comparison between images and/or a combination of images that were created with different signal receivers and different physical units. Thus, effective comparisons can be made between two images that were created using different signal receivers (such as one image created with an information theoretic signal receiver and another image created with a conventional signal receiver, or one image created with a first type of information theoretic signal receiver and a second image created with a second type of information theoretic signal receiver).

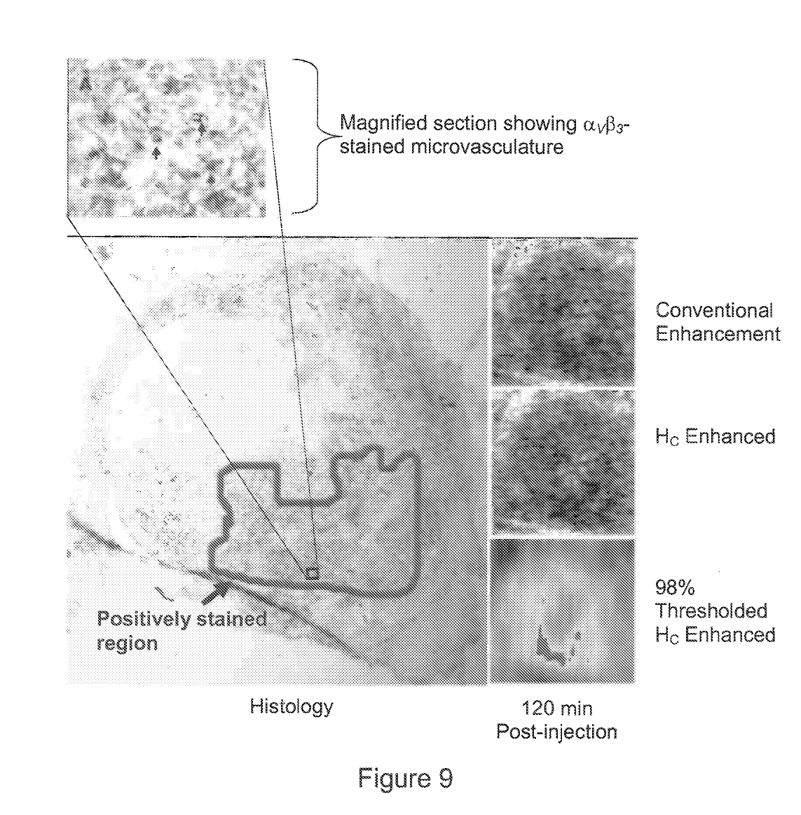

Also, the pixels that are above the brightness threshold (such as the upper 5% of pixels as shown in FIG. 4) can be combined with the pixels of a conventional image, wherein the pixels of the conventional image that are coextensive with the pixels passing the threshold are replaced by the brightest 5% of the information theoretic signal receiver-enhanced pixels. The information theoretic signal receiver-enhanced pixels can then be color-coded to clearly delineate the contrast enhanced area. An example of this is shown in the middle right frame of FIG. 9.

It should be noted that steps 202 through 218 can be repeated over time to generate a plurality of images that would track the enhancement provided by the contrast agent over time. For example, steps 202 through 218 can be performed at 0, 15, 30, 60, and 120 minutes post-injection. Further still, when used to monitor a condition such as angiogenesis, the steps 200 through 218 can be repeated over longer intervals of time to monitor angiogenic development by quantifying changes in the ROI over time.

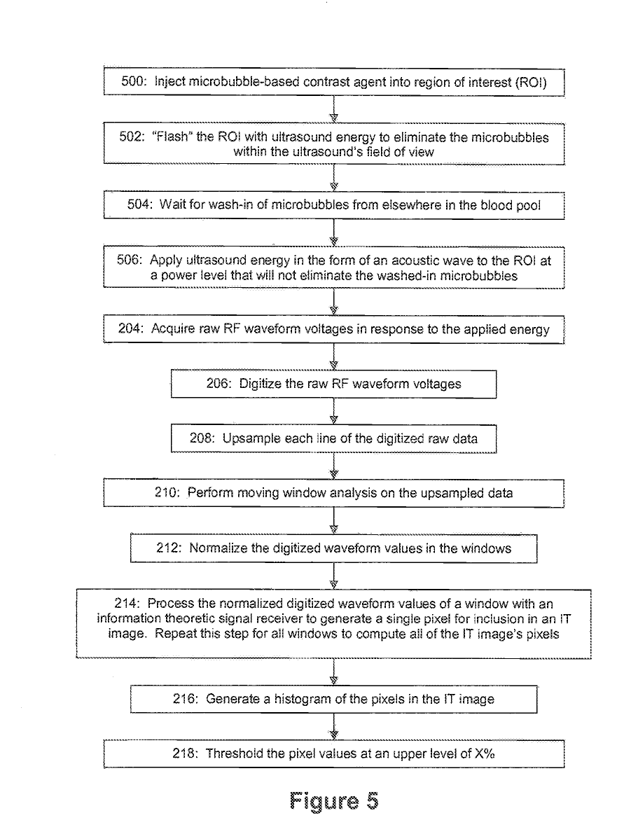

FIG. 5 depicts a methodology that can be used to image a ROI using a microbubble-based contrast agent. At step 500, a microbubble-based contrast agent is injected into a patient's ROI (e.g., 0.12 mL of Optison). At step 502, a high power ultrasound pulse (e.g., MI=2) (or “flash”) is delivered to the region of interest such that substantially all of the microbubbles within the ultrasonic field of view are destroyed. This “flash” establishes a reproducible initial state for the subsequent measurement of the refilling of vasculature, or “wash-in”, by microbubbles from elsewhere in the blood pool. At step 504, the process waits for this wash-in to occur. Subsequently, at step 506, a lower power ultrasound pulse (e.g., MI=0.2) is delivered to the ROI such that not all of the washed-in microbubbles are destroyed as fast. Thereafter, steps 204 through 218 are performed as described in connection with FIG. 2. Preferably, steps 506 and 204-218 are performed at multiple intervals after wash-in has occurred.

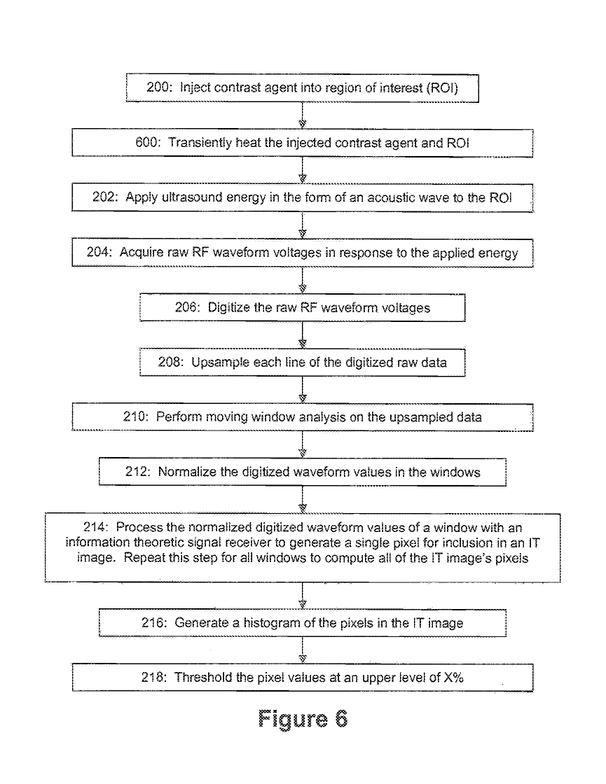

The imaging technique of the present invention can be further enhanced through heating of the contrast agent. FIG. 6 depicts the methodology of FIG. 2, but with an additional step 600 of transiently heating the injected contrast agent and the ROI. Preferably, this heating is a mild non-cavitational heating that heats the ROI to a temperature that is nonlethal for a majority of the cells therein. Temperature changes arising from the application of transient heat preferably fall in a range between 1 and 10 degrees Centigrade. The transient heat can be provided by a focused ultrasound beam, microwave energy, low infrared energy, and even magnetic induction if the contrast agent has iron particles incorporated therein. The time duration for the application of heat to achieve such a temperature change will vary based on the ROI and the equipment being used to provide the heat, however a few seconds of heating would be typical if currently available high frequency ultrasound (HIFU) equipment is used to provide a focused ultrasound beam to heat the injected contrast agent. This aspect of the present invention is believed to be particularly amenable for use with targeted contrast agents such as the PFC nanoparticles of FIG. 3 because the acoustic impedance of PFC nanoparticles is temperature dependent.

As noted above, focused heating can be provided by ultrasound transducers that are capable of delivering a range of energies with programmable frequencies and programmable pulse shapes/trains, such transducers being described in Hughes et al., Acoustic Characterization in Whole Blood and Plasma of Site-Targeted Nanoparticle Ultrasound Contrast Agent for Molecular Imaging, Journal of the Acoustical Society of America, 117(2), pp. 964-72, February 2005; and Hughes et al., Comparison of Ultrasound Scattering Properties of Optison with a Liquid Perfluorocarbon Nanoparticle Contrast Agent, 2001 IEEE Ultrasonics Symposium, 2001; 01CH37263, pp. 1675-78, the entire disclosures of which are incorporated herein by reference. The heating provided by such focused beams can be sufficiently transient to avoid appreciable collateral tissue damage.

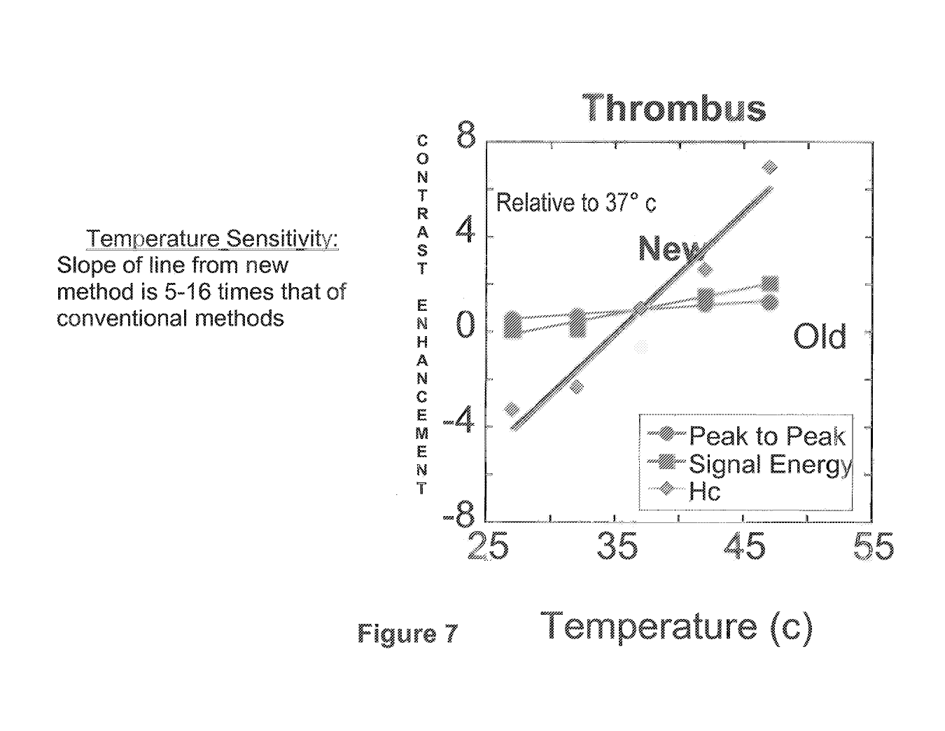

Moreover, as shown in FIG. 7, the contrast enhancement provided by heated PFC nanoparticles when using information theoretic signal receivers is expected to be markedly superior to the contrast enhancement provided by heated PFC nanoparticles when using conventional signal receivers. As FIG. 7 illustrates, the slope of the plot corresponding to information theoretic signal processing is unexpectedly steeper than the slopes of the plots corresponding to conventional signal receivers.

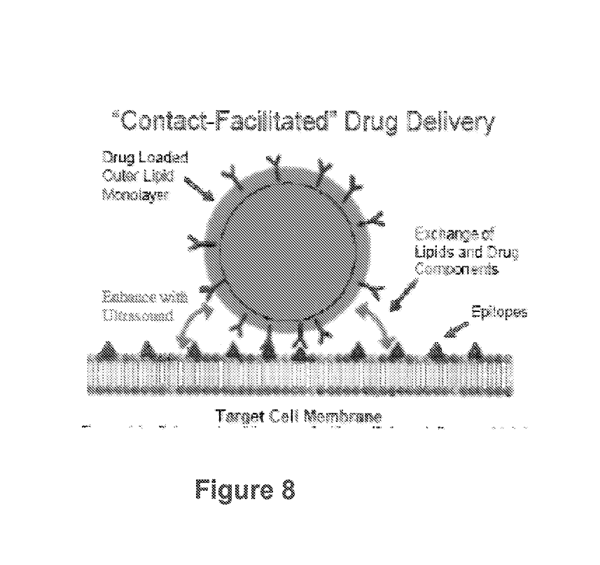

Another useful application for information theoretic signal receivers is to monitor drug delivery through “contact-facilitated” drug delivery. FIG. 8 illustrates the concept of “contact-facilitated drug delivery” wherein a PFC nanoparticle has been loaded with a therapeutic drug. Contact between a target cell membrane and the therapeutic PFC nanoparticle promotes the exchange of lipids and drug components. This contact can be enhanced using ultrasound energy, as has been reported in Crowder et al., Augmented and Selective Delivery of Liquid Perfluorocarbon Nanoparticles to Melanoma Cells with Noncavitational Ultrasound, Proc. IEEE Symposium 2003; 03CH37476C, pp. 532-539, the entire disclosure of which is incorporated herein by reference. It has also been shown in vivo that angiogenesis in atherosclerotic plaque (which is required for plaque growth, as it is for tumor growth) can be inhibited effectively with antiangiogenic drugs formulated into particle lipid membranes. See Winter et al., Molecular Imaging of Angiogenesis in Early-Stage Atherosclerosis with αᵥβ₃-Integrin-Targeted Nanoparticles, Circulation, 2003;108:2270-2274, the entire disclosure of which is incorporated herein by reference.

Ultrasound imaging using information theoretic signal receivers can be used to monitor the attachment of targeted nanoparticles to the targeted epitopes to thereby confirm and quantify the effectiveness of drug delivery. Further still, repeated ultrasound imaging (in conjunction with information theoretic signal receivers) can be performed over time to quantify changes in the targeted ROI over time, thereby providing an indication of the effectiveness of the therapy.

FIG. 9 illustrates the improved contrast enhancement provided by an embodiment of the present invention. Shown at left in FIG. 9 is an αᵥβ₃ stained section of resected tumor from a New Zealand white rabbit, with the stained region (ROI) defined by a trained observer (see the thick red line). The inset at the top of FIG. 9 shows a magnified region inside the ROI that could be used to estimate the density of neovasculature. At the right of FIG. 9 images reconstructed using beamformed RF acquired in vivo from the tumor 120 minutes post-injection of αᵥβ₃ targeted nanoparticles. The plane of ultrasound data acquisition and histological section were the same due to careful resection and cutting. A conventional image is shown in the top right row with the brightest 2% of pixels colored red; this image indicates no enhancement correlated with histology. In the middle of the right row is a conventional image that has been colorized using the brightest 2% of pixels from the H_{c} signal receiver image. In the bottom right row is shown a 98% thresholded H_{c} signal receiver image and colorized at the same 2% level. The enhanced regions in the H_{c}-enhanced and the 98% thresholded H_{c} image are collocated with the region of the excised tumor where αᵥβ₃ expression occurred, thereby indicating the superior ability of the present invention to detect conditions of interest relative to conventional signal receivers in conjunction with contrast agent-based ultrasound imaging.

As previously indicated, the present invention can also be used to detect and monitor conditions such as muscular dystrophy. As described more fully in Exhibit A enclosed herewith, information-theoretic signal receivers can be applied to ultrasound data acquired from a ROI to assess the extent and progressive nature of muscular dystrophy. Preferably, an information-theoretic signal receiver such as the continuous limit Shannon entropy (H_{c}) signal receiver is used to image tissue (which may be a steroid-treated tissue) affected by a muscular dystrophy condition such as MDX. The improved sensitivity of ultrasound images produced with information-theoretic signal receivers can aid in monitoring any therapeutic effects produced in the tissue by the steroid treatments. Moreover, because of the “log-like” scaling of the continuous limit Shannon entropy (H_{c}) signal receiver, ultrasound images generated thereby over time can be effectively compared with each other to monitor therapeutic progress without the need to exactly match the ultrasound machine settings used in previous images. Further still, the information theoretic-based ultrasound images of the ROI can be used in combination with conventional energy-based ultrasound images of the ROI (e.g., a log of the analytic signal magnitude, a 64-point boxcar average of the log energy, etc.) to assess the extent of the muscular dystrophy and the affected tissue's response to therapy.

Additional work by the inventors has indicated that favorable results can also be obtained for assessing muscular dystrophy-affected tissue in humans (e.g. Duchenne Muscular Dystrophy (DMD)) when using clinical ultrasound frequencies (e.g., a 7 MHz ultrasound signal)

While the present invention has been described above in relation to its preferred embodiment, various modifications may be made thereto that still fall within the invention's scope. Such modifications to the invention will be recognizable upon review of the teachings herein. As such, the full scope of the present invention is to be defined solely by the appended claims and their legal equivalents.

What is claimed is:

1. A method for imaging a region of interest (ROI) within a body to obtain an image indicative of a condition of muscular dystrophy affecting the ROI, the method comprising:
applying ultrasound energy to the ROI;
acquiring raw ultrasound data for the ROI in response to the applied energy; and
applying an information theoretic signal receiver to the acquired raw ultrasound data to thereby generate information theoretic data from which an ultrasound image of the ROI can be created, wherein the ultrasound image is indicative of a condition of muscular dystrophy affecting the ROI.

2. The method of claim 2 wherein the ROI comprises tissue that has been treated with a steroid.

3. The method of claim 2 further comprising repeating the method steps over time to determine a progressive condition of the muscular dystrophy affecting the steroid-treated tissue over time.

4. The method of claim 3 wherein the repeating step is performed without requiring exact matching of previous settings for an ultrasound imager when it performs the applying and raw ultrasound data acquiring steps.

5. The method of claim 3 wherein the ROI comprises tissue that has been treated with a steroid, the method further comprising:
applying an energy-based signal receiver to the acquired raw ultrasound data to thereby generate data from which another ultrasound image of the ROI can be created; and
determining the progressive condition of the muscular dystrophy affecting the steroid-treated tissue based on a combination of the information theoretic-based ultrasound image and the energy-based ultrasound image.

6. The method of claim 1 wherein the applying step comprises applying ultrasound energy to the ROI wherein the applied ultrasound energy has a frequency in a range of approximately 2 MHz to approximately 15 MHz.

7. A method for imaging a region of interest (ROI) within a volume to obtain an image indicative of whether a condition of interest exists within the volume, the method comprising:
injecting the region of interest with a contrast agent that is targeted to the condition of interest;
applying ultrasound energy to the ROI;
acquiring ultrasound data for the ROI in response to the applied energy; and
applying an information theoretic signal receiver to the acquired ultrasound data to thereby generate information theoretic data from which an ultrasound image of the ROI can be created, wherein the ultrasound image is indicative of whether the condition of interest exists within the volume.

8. The method of claim 7 wherein the step of applying ultrasound energy comprises applying ultrasound energy to the ROI wherein the applied ultrasound energy has a frequency in a range of approximately 2 MHz to approximately 15 MHz.

9. The method of claim 8 wherein the condition of interest comprises angiogenesis.

10. The method of claim 8 further comprising repeating the ultrasound energy applying step, the acquiring step and the information theoretic signal receiver applying step over time to monitor how the condition of interest progresses over time.

11. The method of claim 10 wherein the contrast agent comprises a targeted contrast agent that has been loaded with a therapeutic drug for contact-facilitated drug delivery, and wherein the repeating step thereby allows the drug delivery to be monitored over time.

12. The method of claim 8 wherein the contrast agent comprises a bubble-based contrast agent.

13. The method of claim 8 wherein the condition of interest comprises tissue affected by muscular dystrophy.

14. The method of claim 7 wherein the acquiring step comprises: (1) receiving raw ultrasound data in response to the applied ultrasound energy and upsampling the raw ultrasound data to stabilize the information theoretic signal receiver when it is applied to the acquired ultrasound data, and (2) performing a moving window analysis on the upsampled data to generate a plurality of windows of upsampled data, and wherein the information theoretic signal receiver applying step comprises applying an information theoretic signal receiver to each generated window of upsampled data.

15. The method of claim 7 wherein the step of applying an information theoretic signal receiver comprises applying at least one member selected from the group consisting of: a Shannon entropy (Hₛ) signal receiver, a continuous limit Shannon entropy (H_{c}) signal receiver, a Renyi entropy (Z) signal receiver, a specific heat analog (Cᵥ) signal receiver, and a thermodynamic energy analog (Eₜₕ) signal receiver to the acquired ultrasound data to thereby generate an ultrasound image that is indicative of whether the condition of interest exists within the volume.

16. The method of claim 7 wherein the ROI comprises a region within a patient's body having a plurality of cells, the method further comprising heating at least a portion of the ROI to a temperature that is nonlethal to a majority of cells within the ROI after the contrast agent has been injected and prior to the ultrasound data acquiring step.

17. The method of claim 16 wherein the heating step comprises applying the heat to the ROI with a heating pulse of ultrasound energy.

18. The method of claim 7 wherein the information theoretic data comprises a plurality of pixels having data values, the method further comprising:
defining a threshold for the information-theoretic data;
selecting pixels of the information theoretic data whose data values are equal to or greater than the threshold; and
generating the ultrasound image by combining the acquired ultrasound data with the selected pixels to create a hybrid ultrasound image.

19. A method for imaging a region of interest (ROI) within a volume to obtain an image indicative of whether a condition of interest exists within the volume, the method comprising:
injecting the ROI with a contrast agent, the contrast agent comprising a plurality of bubbles;
applying a first ultrasound energy to an area within the ROI, wherein the ultrasound energy has sufficient power to eliminate substantially all of the bubbles from the area;
after the area has refilled with a plurality of wash-in bubbles, applying a second ultrasound energy to the area within the ROI, wherein the ultrasound energy has a sufficiently low power to not eliminate the wash-in bubbles;
acquiring raw ultrasound data for the ROI in response to the applied second ultrasound energy;
applying an information theoretic signal receiver to the acquired raw ultrasound data to thereby generate an ultrasound image that is indicative of whether the condition of interest exists within the volume.

20. The method of claim 19 wherein the contrast agent comprises at least one selected from the group consisting of a microbubble-based contrast agent and a nanobubble-based contrast agent, and wherein the step of applying the second ultrasound energy comprises applying second ultrasound energy to the ROI at a clinical frequency.
